(19) United States
(12) Patent Application Publication
(10) Pub. No.: US 2021/0069098 A1
(43) Pub. Date: Mar. 11, 2021

(54) RIBAVIRIN PERFLUBRON EMULSION COMPOSITION FOR TREATING VIRAL DISEASES

(71) Applicant: MT RESEARCH, LLC, SAN DIEGO, CA (US)

(72) Inventor: MATTHEW T. MCLEAY, OMAHA, NE (US)

(21) Appl. No.: 16/950,185

(22) Filed: Nov. 17, 2020

Related U.S. Application Data
(63) Continuation of application No. 12/454,663, filed on May 21, 2009.

(51) Int. Cl. A61K 9/00; A61K 31/198; A61K 45/06; A61K 31/351; A61K 31/7056; A61K 31/215; A61K 38/44

(52) U.S. Cl. CPC A61K 9/008; A61K 31/198; A61K 45/06; C12Y 115/01001; A61K 31/7056; A61K 31/215; A61K 38/446; A61K 31/351

(57) ABSTRACT

The present invention is a novel method using a MegaRibavirin aerosol or a MegaRibavirin combination of therapeutics for the treatment of viral disease particularly the pandemic influenza strains “swine” 2009 HI N1 and H5N1. This invention utilizes Ribavirin in an aerosol Mega Dose (61-161 mg/ml) alone or combined with or without other antivirals, a perfluorocarbon emulsion and anti-inflammatory/anti-oxidants. Where applicable, the perfluorocarbon emulsion may dissolve these agents enabling a depot effect and possible protracted delivery. In addition, perfluorocarbon emulsions have the possible added benefit of oxygen carrying capacity and alveolar nitric oxide sequestration, which may reduce peroxynitrite formation and decrease Influenza severity.

### CROSS-REFERENCE TO RELATED APPLICATIONS

This application is a continuation of co-pending U.S. patent application Ser. No. 12/454,663, filed May 21, 2009, the entire disclosure of which is incorporated herein by reference.

### BACKGROUND OF THE INVENTION

### Field of the Invention

The present invention generally relates to a method of treatment for acute Influenza or other viral diseases. This invention specifically envisions a treatment for Pandemic Influenza such as “swine” 2009 H1N1 or avian H5N1 Influenza but may include any Influenza pandemic strain.

### Description of the Related Art

Ribavirin is an antiviral drug that has activity against a number of DNA and RNA viruses with an uncertain antiviral action. A number of possibilities have been proposed but none are proven and more than one mechanism may be occurring. Ribavirin is a nucleoside antimetabolite that interferes with replication of viral genetic material and also thought to inhibit RNA polymerase. Of notable importance, no significant resistance to Ribavirin has been reported in Influenza including “swine” 2009 H1N1 or H5N1.

Though not effective against all viruses, Ribavirin has broad antiviral action including Influenza, Flavivirus and Hemorrhagic viruses. In the U.S. the oral (capsule or tablet) form of ribavirin is used in combination with interferon drugs for the treatment of hepatitis. An aerosol form has been used to treat primarily respiratory syncytial virus in children.

The primary serious adverse effect of ribavirin is hemolytic anemia. The mechanism for this effect is unknown. The anemia is dose-dependent and maybe compensated by decreasing the dose and/or treatment with agents such as erythropoietin.

Neuraminidase inhibitors (NI) such as Zanamivir and oseltamivir block the enzymatic activity of the surface glycoprotein neuraminidase, which is utilized in releasing the newly formed virions. Zanamivir is one NI that is efficacious for Influenza A and B for both prophylaxis and treatment and is delivered in a dry powder inhaler. However use, as monotherapy may not be optimal as resistance is likely to develop under intense pressure in a pandemic.

Although zanamivir resistant strains were predicted to be “weakened” a recent study using engineered NI resistant H5N1 strains found that H5N1 retained its viral fitness following modification and thus these NI resistant strains would be predicted to cause severe disease (Yen et al J Virology 2007 November; 81(22): 12418-26. Epub 2007 S).

Oxygent™ is a liposome like formula of emulsified perflubron and has been used as a “blood substitute” in Phase I to III studies without “proven” side effects. The small particle size and possible liposome-like delivery makes a perfluorocarbon emulsion such as the perflubron emulsion an ideal agent since other therapeutics possibly both water soluble and non-water soluble may be dissolved in the emulsion as opposed to neat perflubron. Oxygent has been demonstrated to dissolve large amounts of 02 and CO2 and deliver 02 by a diffusion gradient to the tissues reducing the need of allogenic blood transfusions. Further a reduction in metabolic acidosis has been demonstrated using gastric tonometry.

With the addition of a liposome like formula such as a perflubron emulsion, an improved delivery may result. Liposomes have been shown to facilitate delivery of Ribavirin to the tissues especially the lung, liver and spleen (Kende et al Antimicrobial Agents and Chemotherapy June 1985 p 903-907). Thus the combination of Ribavirin with the perflubron emulsion's liposomal like action may serve as a depot allowing a protracted release of drugs.

Partial liquid ventilation (PLV) using neat perflubron has been used in patients with acute respiratory distress syndrome (Kacmarek, R M et al, AmiRespirCritCareMed.2005.0ctober27, ePub ahead of print). These authors performed a phase-III trial that failed to demonstrate a reduction in ventilator days or mortality and there were more complications in the patients given either a “low dose” (10 mUkg) or a “high dose” (20 mL/kg) perflubron that were suggested to be due to trial design and not the product itself. Large tidal volumes, high doses of perflubron and the protocol requirement to disconnect the mechanical ventilator to give the neat perflubron resulted in hypotension, hypoxemia and pneumothorax.

In contrast to PLV, nebulized neat perflubron has been shown to have efficacy for gas exchange in a surfactant depleted animal model (von der Hardt, K, et al, CritiCareMed.2004, May: 32 (5) 1200:6) and is expected to outperform PLV, given that aerosolized perflubron distributes widely in the lungs whereas PLV tends to go to dependent areas. A liposome-like formulation such as perflubron emulsion would be expected to have both the added agents) and less evaporation than with neat perflubron.

A study by MTM Research LLC sponsored by the NIAID under contract to Baylor Medical Center found efficacy of a Ribavirin aerosol with or without a perflubron emulsion dramatically improved survival in a lethal Influenza model. Compared to untreated controls, animals treated with Ribavirin aerosol with or without perflubron emulsion had markedly improved survival: controls had 87¬100% mortality with a mean date of death of 8.3 days while both treated groups had a 85-95% survival. In this study 10 ml aerosol solutions were either Ribavirin 100 mg/ml in water or Ribavirin 100 mg/ml in the perflubron emulsion Oxygent. Treatment was given Q 12 hrs for 3½ days beginning 24 hours after infection. No significant toxicity or mortality differences were observed between the Ribavirin-H20 or Ribavirin-Oxygent treatment aerosol groups or in EC50 in vitro studies. The perflubron emulsion may reduce the alveolar surface tension as well as decrease inflammation by inhibition of cytokines.

These results appear to be an improvement over previously reported High Dose Ribavirin aerosol (HDR) mice studies in which 60 mg/ml aerosol was given for 15 minutes Q 8 hrs in a high mortality model: controls had 100% mortality while treated groups had a 70% survival with treatment initiated 24 hours after infection, Gilbert et al 1992 Antiviral Research 17 (1992) p 33-42).

While the current data reveal no significant change in mortality whether the perflubron emulsion vehicle Oxygent is present further studies are needed. The choice of combinations above may also depend on the pathogenesis of the individual virus.

The above combinations of Ribavirin, other antivirals e.g. zanamivir, perflubron emulsion and antioxidants may prove to be synergistic. In addition to the possible 5-fold increase in drug delivery previously observed with a Ribavirin liposome formula, the perfluorocarbon emulsion would have the added of benefit of oxygen delivery and anti-inflammatory action (Angelova et al 2004 Resuscitation 62 p 89-96, Conley et al Antimicrob Agents Chemotherapy. 1997 June; 41(6): 1288-1292).

Treatment for severe swine 2009 H1N1or H5N1 Influenza or other similar viruses would likely require both anti-viral and anti-inflammatory action. H5N1 Influenza, for instance, has been found to cause severe inflammation in the lung parenchyma as the virus appears to preferentially invade type-2 pneumocytes (Uiprasertkul et al CDC Vol No.7 July 2005) and is suggested to cause massive release of cytokines (Rimmelzwaan et al Avian Dis. 2003; 47(3 Suppl): 931-3). N-acetylcysteine (NAC) and superoxide dismutase (SOD) have also been shown to improve survival in lethal Influenza models and thus would be expected to contribute to efficacy as well.

### SUMMARY OF THE INVENTION

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

The present invention includes a new method for treating acute viral disease. Specific etiologies include Influenza treated by a MegaRibavirin aerosol alone or in a combination with another antiviral, a perfluorocarbon emulsion and an antioxidant.

A short course (less than 2 weeks) of this combination is expected to be needed and have fewer side effects and be very well tolerated compared to current prolonged oral Ribavirin treatment regimens.

Given the current environment with a potential apocalyptic level H5N1 or other Pandemic Influenza such as swine H1N1, there would be very limited options for the potential thousands of patients in hypoxemic respiratory failure. This invention would be utilized along with supplemental oxygen (if needed and available) to treat patients both in a medical or even a non-medical environment.

If prompt treatment with the above invention is administered, severe hypoxemic respiratory failure from Influenza or other viruses may be averted. Only limited medical knowledge and familiarity of equipment would be required to administer these agents when they are nebulized thus making it possible to utilize outside of a medical facility. However, these treatments may be used on non-invasive or mechanical ventilation as well.

### DESCRIPTION OF THE PREFERRED EMBODIMENT

Embodiments are described more fully below with reference to the accompanying figures, which form a part hereof and show, by way of illustration, specific exemplary embodiments. These embodiments are disclosed in sufficient detail to enable those skilled in the art to practice the invention. However, embodiments may be implemented in many different forms and should not be construed as being limited to the embodiments set forth herein. The following detailed description is, therefore, not to be taken in a limiting sense in that the scope of the present invention is defined only by the appended claims.

Ribavirin has a long history of efficacy against viruses notably Influenza, Respiratory Syncytial Virus and Hepatitis.

Example Pandemic Influenza treatments could be exemplified by an aerosol treatment combination such as Ribavirin 1000 mg and Zanamivir 150 mg diluted in 10 ml of sterile saline and given over 30 minutes once or twice daily for 4-7 days. This combination could also include a perflubron emulsion 60% w/v, NAC 20% 2 ml and SOD 20 mg/ml 0.5 ml alone or separate.

By using drugs with different mechanisms and anti-inflammatory agents this may have a substantial advantage over using any drug alone (Ghezzi and Ungheri, 2003 Intern J of Imm and Pharm p 99-101;). However doses of Ribavirin may be used alone 61-161 mg/ml and given over 4 days. Zanamivir 5-20 mg/ml and may be with or without a perfluorocarbon emulsion 1-100% w/v given for 5¬120 minutes up to every 4 hours for up to 2 weeks. Further NAC may be given as 5-20% solution 1-3 ml and SOD 5-25 mg/ml 0.1-3 ml may also be included.

In addition to a new concentration and the use of a novel vehicle the present invention uses a new nebulizer, the Aerotech II, which provides a stable small particle size aerosol for deep deposition delivery, which is advantageous specifically for H5N1 Influenza. However this application is not restricted to any current brand of nebulizers as several Nebulizers are considered possible to use.

Although the invention has been described in language that is specific to certain structures and methodological steps, it is to be understood that the invention defined in the appended claims is not necessarily limited to the specific structures and/or steps described. Rather, the specific aspects and steps are described as forms of implementing the claimed invention. Since many embodiments of the invention can be practiced without departing from the spirit and scope of the invention, the invention resides in the claims hereinafter appended.

What is claimed is:

1. A method of treatment for a patient with influenza comprising delivering to the patient via a nebulizer (a) aerosolized ribavirin at a concentration greater than 61 and less than 161 mg/ml and (b) aerosolized perflubron.

2. The method of claim 1 further comprising delivering to the patient via a nebulizer zanamivir.

3. The method of claim 1 further comprising delivering to the patient via a nebulizer oseltamivir.

4. The method of claim 1 wherein said perflubron is a perflubron emulsion.

5. The method of claim 3 wherein said ribavirin is combined with the perflubron emulsion.

6. The method of claim 1 further comprising delivering to the patient via a nebulizer N acetylcysteine.

7. The method of claim 1 further comprising delivering to the patient via a nebulizer dismutase.

8. The method of claim 1 further comprising the step of administering oxygen simultaneously with said aerosolized perflubron.

9. The method of claim 1 comprising administering the aerosolized ribavirin and aerosolized perflubron to the patient via the nebulizer for about 30 minutes once or twice daily for 4-7 days, wherein said aerosolized composition comprises 1000 mg ribavirin diluted in 10 mL sterile saline and perflubron emulsion at 60% w/v.

10. The method of claim 9 further comprising administering aerosolized 150 mg zanamivir diluted in 10 mL sterile saline, aerosolized 2 mL of 20% w/v N-acetylcysteine, and aerosolized 0.5 mL of 20 mg/mL superoxide dismutase to the patient via the nebulizer.

11. A pharmaceutical composition consisting of aerosolized particles comprising ribavirin and perflubron in sterile saline.

12. The pharmaceutical composition of claim 12 further comprising 15 mg/mL zanamivir.

13. The pharmaceutical composition of claim 12 further comprising N-acetylcysteine.

14. The pharmaceutical composition of claim 12 further comprising superoxide dismutase.

15. The pharmaceutical composition of claim 12, wherein the ribavirin is present at a concentration of 100 mg/mL or 61-161 mg/mL.

16. The pharmaceutical composition of claim 12, wherein the perflubron is a 60% emulsion.

17. A pharmaceutical composition comprising 61 mg/mL-161 mg/mL ribavirin and a 60% perflubron emulsion.

18. The pharmaceutical composition of claim 17 further comprising one or more additional antiviral drugs selected from the group consisting of zanamivir and oseltamivir.

19. The pharmaceutical composition of claim 17 further comprising one or more additional drugs selected from the group consisting of superoxide dismutase and N-acetylcysteine.

20. The pharmaceutical composition of claim 17, wherein the composition is an aerosol.
